(19) United States
(12) Patent Application Publication
(10) Pub. No.: US 2024/0369535 A1
(43) Pub. Date: Nov. 7, 2024

(54) METHODS AND MATERIALS FOR DETERMINING pGC TARGET DOSAGE

(71) Applicant: Mayo Foundation for Medical Education and Research, Rochester, MN (US)

(72) Inventors: Ye Zheng, Rochester, MN (US); Seethalakshmi R. Iyer, Rochester, MN (US); John C. Burnett, Jr., St. Anthony, MN (US); Sasantha J. Sangaralingham, Rochester, MN (US)

(21) Appl. No.: 18/689,132

(22) PCT Filed: Sep. 7, 2022
(86) PCT No.: PCT/US22/76008

§ 371 Date: Mar. 5, 2024

Related U.S. Application Data
(60) Provisional application No. 63/241,484, filed on Sep. 7, 2021.

(51) Int. Cl. G01N 33/50; A61K 38/22

(52) U.S. Cl. CPC G01N 33/5038; A61K 38/2242; G01N 2800/325; G01N 2800/52

(57) ABSTRACT

Methods and materials for assessing therapeutic efficacy with particulate guanylyl cyclase (pGC) receptor stimulators are provided herein. Also provided herein are methods and materials for administering pGC receptor stimulators to mammals based on the assessment.

### CROSS-REFERENCE TO RELATED APPLICATIONS

This application claims benefit of priority from U.S. Provisional Application No. 63/241,484, filed on Sep. 7, 2021. The disclosure of the prior application is considered part of (and is incorporated by reference in) the disclosure of this application.

### STATEMENT OF FEDERALLY SPONSORED RESEARCH

This invention was made with government support under DK103850 awarded by the National Institutes of Health. The government has certain rights in the invention.

This invention was made with government support under HL136340 awarded by the National Institutes of Health. The government has certain rights in the invention.

This invention was made with government support under AG056315 awarded by the National Institutes of Health. The government has certain rights in the invention.

This invention was made with government support under HL132854 awarded by the National Institutes of Health. The government has certain rights in the invention.

This invention was made with government support under HL 158548 awarded by the National Institutes of Health. The government has certain rights in the invention.

### SEQUENCE LISTING

This application contains a Sequence Listing that has been submitted electronically as an XML file named “07039-2082WO1.XML.” The XML file, created on Sep. 7, 2022, is 5,509 bytes in size. The material in the XML file is hereby incorporated by reference in its entirety.

### TECHNICAL FIELD

This document relates to methods and materials for assessing therapeutic efficacy with particulate guanylyl cyclase (pGC) stimulators, and to methods and materials for administering pGC stimulators to mammals based on the assessment.

### BACKGROUND

The heart, endothelium, and kidney produce potent hormones called natriuretic peptides (NPs). These hormones play key roles in the regulation of blood pressure (BP), blood vessel relaxation, excretion of water (diuresis), and excretion of sodium (natriuresis), and they also counteract numerous detrimental systems such as the renin-angiotensin-aldosterone system (RAAS). NPs also have potent anti-fibrotic, anti-inflammatory, anti-remodeling, and immunomodulatory properties.

The favorable and protective biological actions of the NPs are mediated by binding to pGC receptors, also known as natriuretic peptide receptors (NPRs). Atrial natriuretic peptide (ANP) and B-type natriuretic peptide (BNP) bind to the pGC-A receptor (also known as GC-A, NPR-A, or NPR-1), while C-type natriuretic peptide (CNP) binds to the pGC-B receptor (also known as GC-B, NPR-B, or NPR-2). Binding of NPs to their pGC receptors stimulates generation of the signaling second messenger, 3′,5′ cyclic guanosine monophosphate (cGMP). In acute decompensated heart failure (ADHF), ANP and BNP are elevated as a compensatory mechanism to maintain cardiorenal homeostasis. As ANP and BNP are secreted, they bind to the pGC-A receptor to generate cGMP.

### SUMMARY

This document is based, at least in part, on the discovery that the generation of cGMP via pGC stimulation with native NPs in human plasma can be used to establish whether a patient having a cardiovascular, cardiorenal, or cardiometabolic disorder should be treated with a particular pGC receptor target, and in some cases, whether the treatment should be with a higher or lower dose of the pGC target. For example, as described herein, a novel NP/GC-A/cGMP potency assay was designed to assess the biological potency of patients' circulating ANP and BNP ex vivo, to investigate whether ANP and BNP levels in ADHF are of reduced biological potency and should be treated with a higher dose of a pGC targeted therapeutic. This document also is based, at least in part, on the discovery that generation of cGMP via pGC stimulation with patient plasma in the presence or absence of an added pGC receptor target (e.g., a synthetic/recombinantly produced “designer” NP or a small molecule modulator) can provide an excellent indication of the biological effectiveness of the pGC receptor target. It is to be noted that this document also contemplates analogous assays based on NP/GC-B/cGMP.

This document provides methods and materials for identifying and treating mammals that are likely to respond to treatment with, for example, peptide or small molecule pGC-targeted therapeutics. For example, as disclosed herein, the ability to detect cGMP generation from endogenous NPs was demonstrated in pGC-A overexpressing HEK cells cultured with human plasma collected from normal human subjects or patients with human disease (e.g., ADHF). Further, cGMP production was augmented when a NP analogue or small molecule enhancer was added to the human plasma and incubated with the HEK cells overexpressing the pGC-A receptor or the pGC-B receptor. Thus, the assay provided herein can be used to identify patient responsiveness to particular designer NPs or small molecule pGC technologies, which can assist in optimizing and/or guiding pGC/cGMP stimulating therapies.

Having the ability to identify mammals as being likely to respond to a pGC receptor stimulator can allow those mammals to be properly identified and treated in an effective and reliable manner. For example, a pGC receptor stimulator (e.g., MANP, C53, CRRL-408, or MCUF-651) can be used to treat ADHF patients identified as being likely to respond to the pGC receptor stimulator, using the methods disclosed herein.

In general, one aspect of this document features methods for determining whether a mammal having a cardiovascular, cardiorenal, or cardiometabolic disorder is likely to be responsive to treatment with a pGC receptor stimulator. The methods can include, or consist essentially of, (a) contacting, with a first biological fluid sample from the mammal, a first population of cells that express a pGC-A receptor, a pGC-B receptor, or both a pGC-A receptor and a pGC-B receptor, and measuring a first amount of cGMP produced by the first population of cells after the contacting; (b) contacting a second population of the cells that express a pGC-A receptor, a pGC-B receptor, or both a pGC-A receptor and a pGC-B receptor, with a second biological fluid sample from the mammal in combination with the pGC receptor stimulator, and measuring a second amount of cGMP produced by the second population of cells after the contacting; (c) comparing the first amount of cGMP to the second amount of cGMP; and (d) when the second amount of cGMP is at least 5% greater than the first amount of cGMP, classifying the mammal as likely to respond to the pGC receptor stimulator, or when the second amount of cGMP is not at least 5% greater than the first amount of cGMP, classifying the mammal as not likely to respond to treatment with the pGC receptor stimulator. The mammal can be a human. The cardiovascular, cardiorenal, or cardiometabolic disorder can include heart failure (HF), hypertension (HT), cardiorenal syndrome, chronic kidney disease, metabolic syndrome, acute kidney injury, acute kidney disease, or cardiomyopathy. The HF can be ADHF, HF with reduced ejection fraction, or HF with preserved ejection fraction. The HT can be primary HT, uncontrolled HT, resistant HT, or pulmonary HT. The biological fluid sample can be a blood sample or a plasma sample. The methods can include incubating the first and second populations of cells with the first and second biological fluid samples for at least 10 minutes before measuring the first and second amounts of cGMP. The pGC receptor stimulator can be selected from the group consisting of MANP, C53, CRRL-408, MCUF-651, vosoritide, nesiritide, carperitide, cenderitide, and neprilysin inhibitors.

In another aspect, this document features methods for treating a mammal having a cardiovascular, cardiorenal, or cardiometabolic disorder. The methods can include, or consist essentially of, (a) contacting, with a first biological fluid sample from the mammal, a first population of cells that express a pGC-A receptor, a pGC-B receptor, or both a pGC-A receptor and a pGC-B receptor, and measuring a first amount of cGMP produced by the first population of cells after the contacting; (b) contacting a second population of the cells that express a pGC-A receptor, a pGC-B receptor, or both a pGC-A receptor and a pGC-B receptor, with a second biological fluid sample from the mammal in combination with a pGC receptor stimulator, and measuring a second amount of cGMP produced by the second population of cells after the contacting; (c) comparing the first amount of cGMP to the second amount of cGMP; and (d) administering the pGC receptor stimulator to the mammal when the second amount of cGMP is at least 5% greater than the first amount of cGMP. The mammal can be a human. The cardiovascular, cardiorenal, or cardiometabolic disorder can include HF, HT, cardiorenal syndrome, chronic kidney disease, metabolic syndrome, acute kidney injury, acute kidney disease, or cardiomyopathy. The HF can be ADHF, HF with reduced ejection fraction, or HF with preserved ejection fraction. The HT can be primary HT, uncontrolled HT, resistant HT, or pulmonary HT. The biological fluid sample can be a blood sample or a plasma sample. The methods can include incubating the first and second populations of cells with the first and second biological fluid samples for at least 10 minutes before measuring the first and second amounts of cGMP. The pGC receptor stimulator can be selected from the group consisting of MANP, C53, CRRL-408, MCUF-651, vosoritide, nesiritide, carperitide, cenderitide, and neprilysin inhibitors.

In another aspect, this document features methods for treating a mammal having a cardiovascular, cardiorenal, or cardiometabolic disorder. The methods can include, or consist essentially of, administering a pGC receptor stimulator to the mammal, where the mammal was identified being in need of the pGC receptor stimulator based on a method that includes (a) contacting, with a first biological fluid sample from the mammal, a first population of cells that express a pGC-A receptor, a pGC-B receptor, or both a pGC-A receptor and a pGC-B receptor, and measuring a first amount of cGMP produced by the first population of cells after the contacting; (b) contacting a second population of the cells that express a pGC-A receptor, a pGC-B receptor, or both a pGC-A receptor and a pGC-B receptor, with a second biological fluid sample from the mammal in combination with the pGC receptor stimulator, and measuring a second amount of cGMP produced by the second population of cells after the contacting; (c) comparing the first amount of cGMP to the second amount of cGMP; and (d) determining that the second amount of cGMP is at least 5% greater than the first amount of cGMP. The mammal can be a human. The cardiovascular, cardiorenal, or cardiometabolic disorder can include HF, HT, cardiorenal syndrome, chronic kidney disease, metabolic syndrome, acute kidney injury, acute kidney disease, or cardiomyopathy. The HF can be ADHF, HF with reduced ejection fraction, or HF with preserved ejection fraction. The HT can be primary HT, uncontrolled HT, resistant HT, or pulmonary HT. The biological fluid sample can be a blood sample or a plasma sample. The cells can have been incubated with the biological fluid sample for at least 10 minutes before measuring the amount of cGMP. The pGC receptor stimulator can be selected from the group consisting of MANP, C53, CRRL-408, MCUF-651, vosoritide, nesiritide, carperitide, cenderitide, and neprilysin inhibitors.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a pie chart plotting the percentages of ADHF patients that exhibited deficient or elevated levels of plasma ANP and/or BNP.

FIG. 2 is a pie chart plotting the percentages of ADHF patients that exhibited deficient or elevated levels of plasma NT-proANP and/or NT-proBNP.

FIG. 3A is a graph plotting the association of neprilysin (NEP) activity with biologically active ANP and BNP in ADHF patients. FIG. 3B is a graph plotting the correlation of NEP activity with biologically active ANP in ADHF patients. FIG. 3C is a graph plotting the correlation of NEP activity with biologically active BNP in ADHF patients. FIG. 4 is a graph plotting the results of a potency assay carried out to evaluate the efficacy of endogenous plasma ANP and BNP on cGMP production in HEK293 cells overexpressing human pGC-A. The plasma was from healthy subjects (left portion of the graph), ANP and BNP deficient ADHF patients (center portion of the graph), and ADHF patients with elevated ANP and BNP (right portion of the graph), in comparison to concentrations of synthetic ANP alone, synthetic BNP alone, and synthetic ANP+BNP, added in amounts corresponding to the average amounts measured in the plasma being tested. *, P<0.05 with respect to plasma within respective cohort; Ł; P<0.05 with respect to healthy plasma; †, P<0.05 with respect to ADHF plasma with ANP and BNP deficiency.

FIG. 5 is a graph plotting the results of a potency assay conducted to evaluate the potency of MANP for stimulating cGMP production in HEK293 cells overexpressing human GC-A when added to plasma from healthy subjects (left portion of the graph), plasma from ANP and BNP deficient ADHF patients (center portion of the graph), and ADHF patients with elevated ANP and BNP (right portion of the graph). *, P<0.05 vs. vehicle (Veh).

FIG. 6 is a schematic illustrating an augmented NP response in ADHF established using the potency assay provided herein. The potency assay demonstrated an intact NP/GC-A/cGMP system in healthy subjects, mild reduction in potency for ANP and BNP deficient patients, and marked reduction in potency in more severe ADHF.

FIG. 7A shows the structure of MCUF-651. FIG. 7B is a graph plotting cGMP generation in response to MCUF-651 in human GC-A overexpressing HEK293 cells when incubated with human plasma from normal subjects, hypertensive patients, and heart failure patients (N=6/group), in which endogenous ANP and BNP levels were present. *, P<0.05 vs. Veh within each subject group.

FIG. 8A is a graph plotting the results of a potency assay conducted to evaluate the potency of vehicle (Veh) or ANP for stimulating cGMP production in HEK293 cells overexpressing human pGC-A when added to plasma from chronic kidney disease (CKD) patients. FIG. 8B is a graph plotting the results of a potency assay conducted to evaluate the potency of Veh or MANP for stimulating cGMP production in HEK293 cells overexpressing human pGC-A when added to plasma from CKD patients. FIG. 8C is a graph plotting the results of a potency assay conducted to evaluate the potency of Veh or CRRL-408 for stimulating cGMP production in HEK293 cells overexpressing human pGC-A when added to plasma from CKD patients. FIG. 8D is a graph plotting cGMP generation in response to Veh or MCUF-651 in HEK293 cells overexpressing human pGC-A when incubated with human plasma from CKD patients.

FIG. 9A is a graph plotting the results of a potency assay conducted to evaluate the potency of vehicle (Veh) or ANP for stimulating cGMP production in HEK293 cells overexpressing human pGC-A when added to plasma from HT patients. FIG. 9B is a graph plotting the results of a potency assay conducted to evaluate the potency of Veh or MANP for stimulating cGMP production in HEK293 cells overexpressing human pGC-A when added to plasma from HT patients. FIG. 9C is a graph plotting the results of a potency assay conducted to evaluate the potency of Veh or CRRL-408 for stimulating cGMP production in HEK293 cells overexpressing human pGC-A when added to plasma from HT patients. FIG. 9D is a graph plotting cGMP generation in response to Veh or MCUF-651 in HEK293 cells overexpressing human pGC-A when incubated with human plasma from HT patients.

FIG. 10A is a graph plotting the results of a potency assay conducted to evaluate the potency of vehicle (Veh) or ANP for stimulating cGMP production in HEK293 cells overexpressing human pGC-A when added to plasma from ADHF patients. FIG. 10B is a graph plotting the results of a potency assay conducted to evaluate the potency of Veh or MANP for stimulating cGMP production in HEK293 cells overexpressing human pGC-A when added to plasma from ADHF patients. FIG. 10C is a graph plotting the results of a potency assay conducted to evaluate the potency of Veh or CRRL-408 for stimulating cGMP production in HEK293 cells overexpressing human pGC-A when added to plasma from ADHF patients. FIG. 10D is a graph plotting cGMP generation in response to Veh or MCUF-651 in HEK293 cells overexpressing human pGC-A when incubated with human plasma from ADHF patients.

FIG. 11A is a graph plotting cGMP generation in response to vehicle (Veh) or MCUF-651 in HEK293 cells overexpressing human pGC-A when incubated with human plasma from ADHF patients with low circulating ANP and BNP (ANP=19±15 pg/mL; BNP=28±15 pg/mL). FIG. 11B is a graph plotting cGMP generation in response to Veh or MCUF-651 in HEK293 cells overexpressing human pGC-A when incubated with human plasma from ADHF patients with high circulating ANP and BNP (ANP=686±73 pg/mL; BNP=1048±182 pg/mL).

FIG. 12A is a graph plotting the results of a potency assay conducted to evaluate the potency of Veh or CNP for stimulating cGMP production in HEK293 cells overexpressing human pGC-B when added to plasma from ADHF patients. FIG. 12B is a graph plotting the results of a potency assay conducted to evaluate the potency of Veh or C53 for stimulating cGMP production in HEK293 cells overexpressing human pGC-B when added to plasma from ADHF patients. FIG. 12C is a graph plotting the results of a potency assay conducted to evaluate the potency of Veh or CRRL-408 for stimulating cGMP production in HEK293 cells overexpressing human pGC-B when added to plasma from ADHF patients.

### DETAILED DESCRIPTION

This document provides methods and materials for determining whether a pGC stimulator (a molecule or compound that can activate production of cGMP in a cell, such as MANP, C53, CRRL-408, or MCUF-651) is likely to be a useful treatment for a mammal having a cardiovascular, cardiorenal, or cardiometabolic disorder, based on the amount of cGMP produced in response to a biological sample (e.g., a blood sample) from the mammal as compared to the amount of cGMP produced in response to a corresponding sample from the mammal in combination with the pGC stimulator. In some cases, the methods and materials provided herein can be used to determine how much of a pGC stimulator to administer to a mammal, based on how much cGMP a sample from the mammal activates as compared to the amount of cGMP activated by a sample from the mammal in combination with the pGC stimulator. In some cases, the methods provided herein can include administering to the mammal an appropriate dose of a pGC stimulator.

Any appropriate mammal can be assessed and/or treated as described herein. For example, humans, non-human primates, dogs, cats, horses, cows, pigs, sheep, mice, rabbits, and rats can be assessed for their ability to activate cGMP using the methods described herein, and can be treated with a dose of a pGC stimulator based on the result of the assessment. In some cases, the mammal can be a human patient (e.g., a human patient identified as having a cardiovascular disorder such as HF (e.g., ADHF, HF with reduced ejection fraction, or HF with preserved ejection fraction), HT (e.g., primary HT, uncontrolled HT, resistant HT, or pulmonary HT), a cardiorenal disorder, a cardiometabolic disorder, cardiorenal syndrome, or cardiomyopathy, or identified as having chronic kidney disease, metabolic syndrome, acute kidney injury, or acute kidney disease.

Any appropriate sample from a mammal can be assessed as described herein (e.g., assessed to determine whether a pGC receptor stimulator can increase the sample's ability to activate cGMP). In some cases, biological samples such as fluids (e.g., whole blood, plasma, serum, urine, cerebrospinal fluid, synovial fluid, or saliva) can be obtained from a mammal and used to determine the mammal's ability to stimulate cGMP production using the methods provided herein. For example, plasma can be obtained and used to determine a relative level of cGMP activation with and without a pGC receptor stimulator. The relative level then can be used to determine whether the mammal is likely to respond to treatment with the pGC stimulator. In some cases, the relative level can be used to determine an appropriate dosage of a pGC target.

The methods provided herein can include using cells (e.g., transgenic cells) that overexpress the pGC-A and/or pGC-B receptor. Any suitable type of cells can be used. In some cases, for example, human embryonic kidney (HEK) 293 cells that overexpress pGC-A and/or pGC-B can be used in the methods provided herein. In some cases, COS cells (a fibroblast-like cell line derived from monkey kidney tissue) that overexpress pGC-A and/or pGC-B can be used in the methods provided herein. Cells that overexpress pGC-A or pGC-B can be prepared by, for example, stably transfecting a suitable cell line (e.g., HEK293 cells or COS cells) with a nucleic acid encoding a pGC-A or pGC-B polypeptide. Plasmids that contain sequences encoding pGC-A or pGC-B are commercially available (e.g., from Origene, Rockville, MD; Cat. Nos. RG209267 and RG220459, respectively). Nucleotide and amino acid sequences for pGC-A are available in GENBANK® under Accession No. NM_000906 (e.g., version NM_000906.4). Nucleotide and amino acid sequences for pGC-B are available in GENBANK® under Accession No. NM_003995 (e.g., version NM_003995.4).

The methods provided herein can include contacting cells that overexpress pGC-A and/or pGC-B with a biological fluid sample obtained from a mammal (e.g., a mammal having a cardiovascular disorder such as HF, ADHF, or HT). The cells can be incubated with the biological fluid sample for any appropriate length of time (e.g., about 1 to about 3 minutes, about 2 to about 5 minutes, about 3 to about 8 minutes, about 5 to about 10 minutes, about 7 to about 12 minutes, about 10 to about 15 minutes, about 15 to about 20 minutes, at least about 20 minutes, about 1 minute, about 2 minutes, about 3 minutes, about 4 minutes, about 5 minutes, about 6 minutes, about 7 minutes, about 8 minutes, about 9 minutes, about 10 minutes, about 15 minutes, or about 20 minutes).

After the cells are incubated with a biological sample for a suitable length of time, the amount of cGMP produced by the cells can be determined. Any appropriate method can be used to measure an amount of cGMP. In some cases, for example, an enzyme-linked immunosorbent assay (ELISA) can be used. Kits for carrying out an ELISA to measure cGMP are commercially available (e.g., from Enzo Life Sciences, Farmingdale, NY. Other methods also can be used to determine the amount of cGMP produced by cells contacted with a biological sample (e.g., radioimmunoassay (RIA) or mass spectrometry).

The methods provided herein also can include comparing (a) a measured amount of cGMP produced by a first population of cells contacted with a first aliquot of the biological sample from the mammal to (b) an amount of cGMP produced by a second population of the cells that were treated with a second aliquot of the mammal's biological sample and a pGC receptor stimulator. The second population of cells typically are the same type of cells that were contacted with the biological sample without the pGC receptor stimulator, and can include about the same number of cells as the population that was contacted with the biological sample without the pGC stimulator.

The relative levels of cGMP produced by the first and second populations of cells can indicate whether the mammal is likely to respond to treatment with the pGC stimulator (also referred to as a “pGC receptor stimulator” or a “pGC target”). For example, if the amount of cGMP produced by the cells contacted with the biological fluid sample and the pGC receptor stimulator is at least 5% greater (e.g., at least 10%, at least 15%, at least 20%, at least 25%, at least 30%, at least 40%, or at least 50% greater) than the amount of cGMP produced by the cells contacted with the biological fluid sample alone (without the pGC receptor stimulator), the method can include determining that the mammal is likely to respond to treatment with the pGC receptor stimulator; in some cases, such a response can indicate that a lower dose of the pGC receptor stimulator can be used. Alternatively, if the amount of cGMP produced by the cells contacted with the biological fluid sample and the pGC receptor stimulator is not at least 5% greater than the amount of cGMP produced by the cells contacted with the biological fluid sample alone, the method can include determining that the mammal is not likely to respond to treatment with the pGC receptor stimulator; in some cases, such a response can indicate that a higher dose of the pGC receptor stimulator may be needed or useful.

In some cases, the relative levels can indicate whether a higher or lower dosage of the pGC stimulator should be administered to the mammal from which the biological sample was obtained. For example, if the amount of cGMP produced by the cells contacted with the biological fluid sample and the pGC receptor stimulator is relatively low (e.g., less than 5%, such as about 1% to about 3%, about 2% to about 4%, about 3% to about 5%, about 1% to about 2%, about 2% to about 3%., about 3% to about 4%, about 4% to about 5%, less than 4%, less than 3%, less than 2%, or less than 1% greater than the amount of cGMP produced by the cells contacted with the biological fluid sample alone), then a higher dosage of the pGC stimulator can be administered to the mammal from which the biological sample was obtained. Conversely, if the amount of cGMP produced by the cells contacted with the biological fluid sample and the pGC receptor stimulator is relatively high (e.g., at least 5%, such as about 5% to about 10%, about 10% to about 15%, about 15% to about 20%, at least 10%, at least about 15%, at least 20%, at least 30%, at least 40%, or at least 50% greater than the amount of cGMP produced by the cells contacted with the biological fluid sample alone, then a lower dosage of the pGC stimulator can be administered to the mammal from which the biological sample was obtained

In some cases, the relative levels of cGMP produced by two or more populations of cells treated with two or more different doses of a pGC receptor stimulator can indicate what dosage of the stimulator (of those tested) might be most appropriate. For example, if a biological sample from a patient elicits a strong response to a drug at a relatively high dose, then the initial appropriate starting dose may be determined to be lower. If a biological sample from a patient elicits a weak response to a drug at a relatively low dose, then the initial appropriate starting dose may be determined to be higher.

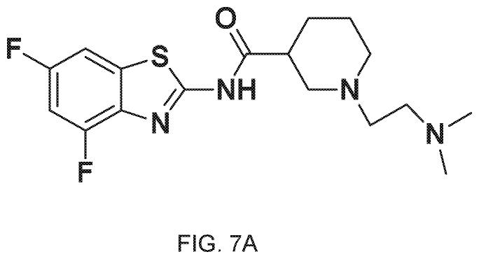

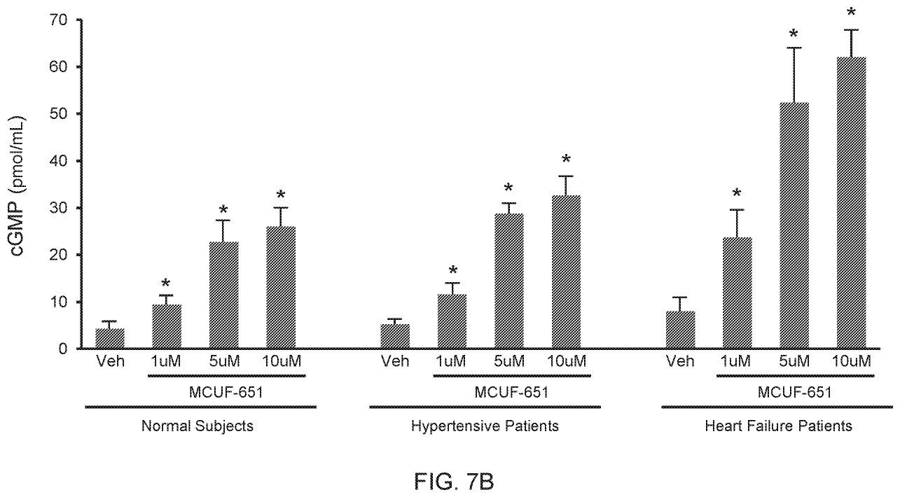

In some cases, the methods provided herein also can include administering to a mammal a pGC stimulator, where the likelihood of response to the pGC stimulator was determined as described herein. Any appropriate pGC stimulator can be administered, including any biologic or small molecule that targets the pGC-A or pGC-B receptor. In some cases, for example, the pGC stimulator can be MANP, a polypeptide having the amino acid sequence SLRRSSCFGGRMDRIGAQSGLGCNSFRYRITAREDKQGWA (SEQ ID NO:1). In some cases, the pGC stimulator can be CRRL-408, a polypeptide that is a dual pGC-A and pGC-B activator and has the amino acid sequence KYKGANKKGL SKGCFGLKLDRIGSMSGLGCPSLRDPRPNAPSTSA (SEQ ID NO:3). In some cases, the pGC stimulator can be C53, a polypeptide that is a pGC-B activator and has the amino acid sequence DLRVDTKSRAAWARLLQEHPNARKYKGANKKGLSKGCFG LKLDRIGSMSGLGC (SEQ ID NO:5). In some cases, the pGC stimulator can be a small molecule, such as MCUF-651 (shown in FIG. 7B and discussed in further detail in Example 2 herein). In some cases, the pGC stimulator can be vosoritide, nesiritide, carperitide, cenderitide, or a neprilysin inhibitor.

In some cases, the methods provided herein can include administering a pGC receptor stimulator that is a variant of the MANP, CRRL-408, or C53 amino acid sequence set forth in SEQ ID NO:1, SEQ ID NO:3, or SEQ ID NO:5, respectively. For example, a method can include administering a MANP polypeptide containing the entire amino acid sequence set forth in SEQ ID NO:1, except that the amino acid sequence can contain from one to five (e.g., five, four, three, two, one, one to five, one to four, one to three, or one to two) amino acid additions, subtractions, and substitutions, or modifications. For example, a polypeptide can contain the amino acid sequence set forth in SEQ ID NO:1 with one, two, three, four, or five single amino acid residue additions, subtractions, or substitutions. In some cases, a polypeptide can contain the amino acid sequence set forth in SEQ ID NO:3 with one, two, three, four, or five single amino acid residue additions, subtractions, or substitutions. In some cases, a polypeptide can contain the amino acid sequence set forth in SEQ ID NO:5 with one, two, three, four, or five single amino acid residue additions, subtractions, or substitutions. Any amino acid residue set forth in SEQ ID NO:1, SEQ ID NO:3, or SEQ ID NO:5 can be subtracted, and any amino acid residue (e.g., any of the 20 conventional amino acid residues or any other type of amino acid such as ornithine or citrulline) can be added to or substituted within the sequence set forth in SEQ ID NO:1, SEQ ID NO:3, or SEQ ID NO:5. The majority of naturally occurring amino acids are L-amino acids, and naturally occurring polypeptides are largely comprised of L-amino acids. D-amino acids are the enantiomers of L-amino acids. In some cases, a polypeptide as provided herein can contain one or more D-amino acids. In some embodiments, a polypeptide can contain chemical structures such as ε-aminohexanoic acid; hydroxylated amino acids such as 3-hydroxyproline, 4-hydroxyproline, (5R)-5-hydroxy-L-lysine, allo-hydroxylysine, and 5-hydroxy-L-norvaline; or glycosylated amino acids such as amino acids containing monosaccharides (e.g., D-glucose, D-galactose, D-mannose, D-glucosamine, and D-galactosamine) or combinations of monosaccharides.

Variant polypeptides having one or more amino acid additions, subtractions, or substitutions relative to the MANP, CRRL-408, and C53 amino acid sequences set forth in SEQ ID NO:1, SEQ ID NO:3, and SEQ ID NO:5, respectively can be prepared and modified using any appropriate method. In some cases, amino acid substitutions can be made by selecting substitutions that do not differ significantly in their effect on maintaining (a) the structure of the peptide backbone in the area of the substitution, (b) the charge or hydrophobicity of the molecule at the target site, or (c) the bulk of the side chain. For example, naturally occurring residues can be divided into groups based on side-chain properties: (1) hydrophobic amino acids (norleucine, methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Non-limiting examples of useful conservative substitutions can include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenyalanine, glycine for proline, threonine for serine, serine for threonine, tyrosine for tryptophan, phenylalanine for tyrosine, and/or leucine for valine.

Further examples of conservative substitutions that can be made at any position within a MANP, CRRL-408, or C53 polypeptide are set forth in TABLE 1.

TABLE 1
Examples of conservative amino acid substitutions

| | | Typical |
|---|---|---|
| Original | | preferred |
| Residue | Exemplary substitutions | substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn | Asn |
| Glu | Asp | Asp |
| Gly | Pro | Pro |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, Gln, Asn | Arg |
| Met | Leu, Phe, Ile | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleucine | Leu |

In some cases, a variant polypeptide can include one or more non-conservative substitutions. Non-conservative substitutions typically entail exchanging a member of one of the classes described above for a member of another class. Such production can be desirable to provide large quantities or alternative embodiments of such compounds. Whether an amino acid change results in a functional polypeptide can readily be determined by assaying the specific activity of the peptide variant using, for example, methods disclosed herein.

A pGC stimulator can be administered to a mammal in any appropriate amount, by any appropriate route (e.g., orally, subcutaneously or intravenously), over any appropriate length of time (e.g., one administration or multiple administrations over a period of days, weeks, or months), such that the stimulator is effective to reduce one or more symptoms or effects of a cardiovascular disorder in the mammal.

A pharmaceutical compositions containing a pGC stimulator can be administered by any appropriate method, depending upon whether local or systemic treatment is desired. Administration can be, for example, parenteral (e.g., by subcutaneous, intrathecal, intraventricular, intramuscular, or intraperitoneal injection, or by intravenous (i.v.) drip), oral, topical (e.g., transdermal, sublingual, ophthalmic, or intranasal), or pulmonary (e.g., by inhalation or insufflation of powders or aerosols), or can occur by a combination of such methods. Administration can be rapid (e.g., by injection) or can occur over a period of time (e.g., by slow infusion or administration of slow release formulations).

In some cases, a pGC stimulator (e.g., a composition that contains a pGC stimulator) can be administered subcutaneously at a dose of about 1 ng pGC stimulator/kg to about 1 mg pGC stimulator/kg of body mass (e.g., about 10 ng pGC stimulator/kg to about 500 μg pGC stimulator/kg, about 20 ng pGC stimulator/kg to about 300 μg pGC stimulator/kg, about 100 ng pGC stimulator/kg to about 100 μg pGC stimulator/kg, about 300 ng pGC stimulator/kg to about 5 μg pGC stimulator/kg, or about 500 ng pGC stimulator/kg to about 2 μg pGC stimulator/kg) of body mass, although other dosages also may provide beneficial results.

In some cases, a pGC stimulator can be administered intravenously at a dose of, for example, about 50 pg pGC stimulator/kg/minute to about 50 μg pGC stimulator/kg/minute (e.g., about 0.5 ng pGC stimulator/kg/minute to about 5 μg pGC stimulator/kg/minute, about 1 ng pGC stimulator/kg/minute to about 1 μg pGC stimulator/kg/minute, about 2 ng pGC stimulator/kg/minute to about 500 ng pGC stimulator/kg/minute, about 5 ng pGC stimulator/kg/minute to about 200 ng pGC stimulator/kg/minute, or about 10 ng pGC stimulator/kg/minute to about 100 ng pGC stimulator/kg/minute).

In some cases, a pGC stimulator can be administered orally at a dose of, for example, about 5 μg to about 1 g (e.g., about 10 μg to about 500 mg, about 50 μg to about 250 mg, about 100 μg to about 100 mg, about 500 μg to about 50 mg, about 1 mg to about 10 mg, or about 2 mg to about 5 mg).

A pGC receptor stimulator or a composition containing a pGC receptor stimulator can be administered once or more than once. When administered more than once, the frequency of administration can range from about two times a day to about once every other month (e.g., four times a day, three times a day, twice a day, once a day, three to five times a week, about once a week, about twice a month, about once a month, or about once every other month). In addition, the frequency of administration can remain constant or can be variable during the duration of treatment. Various factors can influence the actual frequency of administration used for a particular application. For example, the effective amount, duration of treatment, route of administration, and severity of condition may require an increase or decrease in administration frequency.

The methods provided herein can include administering to a mammal an effective amount of a pGC receptor stimulator (e.g., MANP, C53, CRRL-408, or MCUF-651). As used herein, the term “effective amount” is an amount of a molecule or composition that is sufficient to reduce a symptom or effect of a cardiovascular, cardiorenal, or cardiometabolic disorder. In some embodiments, an “effective amount” of a pGC receptor stimulator can be an amount of the pGC receptor stimulator that is sufficient to reduce the occurrence of one or more symptoms of cardiovascular, cardiorenal, and/or cardiometabolic disease by at least 10% (e.g., at least 10%, at least 15%, at least 20%, at least 25%, at least 30%, at least 40%, at least 50%, at least 60%, at least 70%, at least 75%, at least 80%, at least 85%, at least 90%, at least 95%, at least 99%, or 100%). In some cases, for example, an “effective amount” of a pGC receptor stimulator can be an amount of the pGC receptor stimulator that reduces blood pressure, preserves or enhances glomerular filtration rate (GFR), reduces proteinuria, increases natriuresis, reduces cardiac filling pressure, preserves or enhances systolic function, preserves or enhances diastolic function, reduces or prevents cardiac hypertrophy, reduces or prevents cardiac dilatation, reduces or prevents acute kidney injury (AKI), inhibits the aldosterone/renin-angiotensin-aldosterone system, or any combination thereof, in a treated mammal (e.g., as compared to the blood pressure, GFR, proteinuria, natriuresis, cardiac filling pressure, systolic function, diastolic function, cardiac hypertrophy, cardiac dilatation, AKI, and/or aldosterone/renin-angiotensin-aldosterone system in the mammal prior to administration of the pGC receptor stimulator or without administration of the pGC receptor stimulator, or in a control, untreated mammal). In some cases, for example, an “effective amount” of a pGC receptor stimulator as provided herein can be an amount that reduces a symptom of cardiovascular disease in a treated mammal by at least 10% as compared to the level of the symptom in the mammal prior to administration of the pGC receptor stimulator or without administration of the pGC receptor stimulator, or as compared to the level of the symptom in a control, untreated mammal. The presence or extent of such symptoms can be evaluated using any appropriate method. In some cases, an “effective amount” of a pGC receptor stimulator provided herein can be an amount that reduces blood pressure in a mammal identified as having hypertension by at least 10% (e.g., at least 15%, at least 20%, at least 25%, at least 30%, at least 40%, or at least 50%) as compared to the blood pressure in the mammal prior to administration of the pGC receptor stimulator or without administration of the pGC receptor stimulator, or as compared to the level of the symptom in a control, untreated mammal.

In some cases, the amount and frequency of administration for a pGC receptor stimulator administered to a mammal can be titrated in order to, for example, identify a dosage that is most effective to treat a cardiovascular, cardiorenal, or cardiometabolic disease while having the least amount of adverse effects. For example, an effective amount of a composition can be any amount that reduces blood pressure, preserves or enhances GFR, reduces proteinuria, increases natriuresis, reduces cardiac filling pressure, preserves or enhances systolic function, preserves or enhances diastolic function, reduces or prevents cardiac hypertrophy, reduces or prevents cardiac dilatation, reduces or AKI, inhibits the aldosterone/renin-angiotensin-aldosterone system, or any combination thereof, in a mammal without having significant toxicity in the mammal. If a particular mammal fails to respond to a particular amount, then the amount can be increased by, for example, two-fold, three-fold, five-fold, or ten-fold. After receiving this higher concentration, the mammal can be monitored for both responsiveness to the treatment and toxicity symptoms, and adjustments in the dosage can be made accordingly. The effective amount can remain constant or can be adjusted as a sliding scale or variable dose depending on the mammal's response to treatment.

The frequency of administration can be any frequency that reduces a symptom of cardiovascular disease within a mammal without producing significant toxicity in the mammal. For example, the frequency of administration can be from about four times a day to about once every other month, or from about once a day to about once a month, or from about once every other day to about once a week. In addition, the frequency of administration can remain constant or can be variable during the duration of treatment. As with the effective amount, various factors can influence the actual frequency of administration used for a particular application. For example, the effective amount, duration of treatment, route of administration, and severity of the condition may require an increase or decrease in administration frequency.

An effective duration of administration can be any duration that reduces a symptom of a cardiovascular, cardiorenal, or cardiometabolic disease within a mammal without producing significant toxicity in the mammal. The effective duration can vary from one to several days, to several weeks, months, or years. In general, the effective duration can range in duration from several days to several months. For example, an effective duration can range from about one to two weeks to about 36 months. Prophylactic treatments can be typically longer in duration and may last throughout an individual mammal's lifetime. Multiple factors can influence the actual effective duration used for a particular treatment or prevention regimen. For example, an effective duration can vary with the frequency of administration, amount administered, route of administration, and severity of the condition.

After administering a pGC receptor stimulator to a mammal, the mammal can be monitored to determine whether or not the cardiovascular, cardiorenal, or cardiometabolic disease has improved. For example, a mammal can be assessed after treatment to determine whether or not one or more symptoms of the disease have decreased. Any suitable method can be used to assess improvements in function. If a mammal fails to respond to a particular dose, then the amount can be increased by, for example, two-fold, three-fold, five-fold, or ten-fold. After receiving this higher concentration, the mammal can be monitored for both responsiveness to the treatment and toxicity symptoms, and adjustments made accordingly. The effective amount can remain constant or can be adjusted as a sliding scale or variable dose depending on the mammal's response to treatment.

The invention will be further described in the following examples, which do not limit the scope of the invention described in the claims.

### EXAMPLES

### Example 1—Reduced NP Production and Potency in Human ADHF, and an Ex Vivo NP/GC-A/cGMP Potency Assay

In ADHF, ANP and BNP are elevated as a compensatory mechanism to maintain cardiorenal homeostasis. As ANP and BNP are secreted, they bind to GC-A to generate cGMP. In ADHF, a subpopulation of patients are deficient in ANP, which is consistent with reports of BNP deficiency in hospitalized HF patients (Reginauld et al., JACC Hear. Fail. 7:891-898, 2019; and Bachmann et al., JACC Hear. Fail. 9:192-200, 2021).

Studies were conducted to determine whether the ANP and BNP produced in ADHF patients possess reduced potency in GC-A activation, thereby over-estimating the biological potency of elevated endogenous cardiac NP levels. In addition, a NP/GC-A/cGMP potency assay was developed to assess the biological potency of patients' circulating ANP and BNP ex vivo, to assess the potency of ANP and BNP in ADHF.

### Methods

Study Population: Healthy/normal volunteers (N=47) having neither cardiovascular disease nor metabolic disease and not taking cardiovascular medications were recruited, as were ADHF hospitalized patients (N=107). Clinical presentation satisfying Framingham Heart Study criteria was used to confirm ADHF diagnosis (McKee et al., N. Engl. J. Med.285:1441-1446, 1971). In addition to performing routine clinical examination and obtaining medical history, blood was obtained for ANP, BNP, N-terminal pro-atrial natriuretic peptide (NT-proANP), N-terminal pro-B-type natriuretic peptide (NT-proBNP), cGMP, and neprilysin (NEP) activity analyses within 72 hours of hospitalization. Body mass index (BMI), ejection fraction (EF), and estimated glomerular filtration rate (eGFR) were calculated or obtained from electronic medical records.

Determination of Cutoffs: Healthy/normal cohort cutoffs (TABLE 1) were created for ANP, NT-proANP, BNP, and NT-proBNP by using the 95th percentile of NP levels within the healthy cohort. The cutoffs were applied to the newly obtained ADHF cohort in order to differentiate the ADHF patients into either having a low (deficient) ANP or BNP level in ADHF, which was less than or equal to the cutoff and was consistent with an impaired hormonal response in ADHF, or having elevated ANP and/or BNP levels, deemed as having hormone levels greater than the NP cutoff.

TABLE 1
Healthy cohort cutoffs

| NP | Cutoff | |
|---|---|---|
| ANP | 48.1 | pg/mL |
| NT-proANP | 163.6 | pg/mL |
| BNP | 107 | pg/mL |
| NT-proBNP | 219 | pg/mL |

Plasma Assays: Blood samples were centrifuged at 4° C. and 2500 rpm for 10 minutes, and the plasma was stored in 1 mL aliquots at −80° C. until further analysis.

ANP: Plasma ANP was measured using a radioimmunoassay described elsewhere (Burnett et al., Science 231:1145-1147, 1986). Standards were obtained from Phoenix Pharmaceuticals (Burlingame, CA). 100 μL of standards and plasma samples were incubated with 100 μL of diluted (1:50,000) anti-human ANP at 4° C. After 18 hours, 100 μL (10,000 counts) of I¹²⁵-labelled ANP was added and incubated at 4° C. Following an additional 18 hours of incubation, a second antibody was added to all samples to separate the free and bound fractions. Samples were centrifuged, the free fraction was aspirated, and the bound fraction was counted on a gamma counter. A standard curve was generated and used to calculate the concentrations of the unknown samples and reported in pg/mL. The range of the standard curve was 4.9-1250 pg/mL. Inter-and intra-assay variability were 9% and 6%, with recovery being 81±2%. Crossreactivity was <1% with NT-proANP, BNP, CNP, endothelin (ET), and adrenomedullin (ADM).

NT-proANP: NT-proANP was measured using a commercially available radioimmunoassay obtained from Phoenix Pharmaceuticals (Burlingame, CA) (Lerman et al., Lancet 341:1105-1109, 1993). Standards and unknown samples (100 μL each) were incubated with 100 μL rabbit-anti human NT-proANP at 4° C. overnight. After 18 hours of incubation, 100 μL of I¹²⁵ labelled antigen was added and incubated overnight at 4° C. Following an additional 18 hours of incubation, a second antibody was added to all samples to separate the free and bound fractions. Samples were centrifuged, the free fraction was aspirated, and the bound fraction was counted on a gamma counter. Concentrations were calculated using a standard curve ranging between 5-1280 pg/mL, with recovery being 78%, and inter-and intra-assay variability being 24% and 7% respectively. There was no cross-reactivity with ANP, BNP, CNP, or NT-proANP 56-92.

BNP: BNP was assayed using a two-site immunoenzymatic sandwich assay (Biosite Inc, Alere, France) (McKie et al., Hypertension 47:874-880, 2006) and a Beckman Coulter Dxl 800. Samples were added to a reaction vessel with a mouse monoclonal anti-human BNP antibody-alkaline phosphatase conjugate and paramagnetic particles coated with mouse Omniclonal anti-human BNP antibody. BNP in human plasma bound to the immobilized anti-BNP on the solid phase, while the mouse anti-BNP conjugate reacted specifically with bound BNP. After incubation in a reaction vessel, materials bound to the solid phase were held in a magnetic field while unbound materials were washed away. A chemiluminescent substrate, Lumi-Phos* 530, was added to the reaction systems for in vitro quantitative measurement of BNP. Light generated by the reaction was measured with a luminometer. The light production was directly proportional to the concentration of BNP in each sample. The amount of analyte in the sample was determined from a stored, multi-point calibration curve.

NT-proBNP: NT-proBNP was measured using an Elecsys NT-proBNP electrochemiluminescence immunoassay (Roche Diagnostics, Indianapolis, Indiana). Inter and intra-assay variation for NT-proBNP were 3.1% and 2.5%, respectively.

cGMP: Plasma cGMP was measured using an ELISA (Enzo Life Sciences, Farmingdale, NY). Samples and standards (100 μL each) were incubated with cGMP conjugate (50 μL) and cGMP antibody (50 μL) at room temperature for 2 hours at 500 rpm. The plates were washed 4 times and 200 μL of pNPP substrate was added to each well, followed by incubation for 1 hour at room temperature. The reaction was stopped by adding stop solution and absorbance was read at 405 nm and corrected for plate imperfections at 570 nm using the SpectramaxM2. A standard curve was generated using the Softmaxpro software and unknown concentrations were extrapolated based on the standard curve. Samples exceeding the standard curve were diluted as needed in assay buffer and corresponding dilution factors were applied to extrapolate final concentrations. The standard curve range was from 0.16-500 pmol/mL. The CV % at EC₅₀ was 6.6%.

Neprilysin Activity: NEP activity assay was performed using a SENSOLYTE® 520 Neprilysin Assay Kit (Anaspec, Fremont, CA). This kit employs an internally quenched 5-FAM/QXL® FRET substrate for the detection of NEP activity. The enzyme cleaves the FRET substrate into two separate fragments resulting in the release of 5-FAM fluorescence, which can be monitored at an excitation/emission of 490/520 nm. The long wavelength fluorescence of 5-FAM experiences less interference by the auto fluorescence of components in biological samples and test compounds. Briefly, plasma samples (50 μL) were incubated in the presence of the aforementioned FRET-substrate (as per the manufacturer's instructions) for 30 minutes. At the end of incubation, the plate was read using a Spectramax at an excitation/emission of 490/520 nm. NEP supplied with the kit was used as a positive control for the assay. The standard curve was generated using 1 mM fluorescence standard solution provided with the kit. The standard curve ranged from 0.04 μM to 5.0 μM. NEP Activity was calculated as the amount of fluorescent substrate measured in each sample formed over an incubation time of 30 minutes (nM/mL/min). The inter-and intra-assay variability were 2.5% and 0.6%, respectively.

In vitro Cell Culture: HEK 293 cells were stably transfected with human GC-A using LIPOFECTAMINE™ (Invitrogen, Grand Island, NY) (Chen et al., Am J Physiol Regul Integr Comp Physiol. 318(4):R669-R676, 2020). Transfected cells (HEK/GCA) were maintained in Dulbecco Modified Eagle medium (DMEM) supplemented with 10% FBS, and 250 μg/ml G418. Cells in 48-well plates were grown to 80-90% confluency and then incubated in Hanks Balanced Salt Solution (HBSS, Invitrogen) containing 20 mM HEPES, 0.1% BSA and 0.5 mM 3-isobutyl-1-methylxanthine (IBMX, Sigma).

NP/GC-A/cGMP Potency Assay: To evaluate the potency of ANP and BNP in normal/healthy subjects and ADHF patients, an ex-vivo NP/GC-A/cGMP potency assay was developed. A stable transfected cell line of HEK293 cells engineered to overexpress human GC-A was grown in 48-well plates to 80-90% confluency. Plasma samples from healthy volunteers (N=4), ANP and BNP deficient ADHF patients (N=4), and ADHF patients with elevated ANP and BNP (N=4) were diluted 10× with HBSS and incubated with the HEK/GC-A cells for 10 minutes at 37° C. Meanwhile, synthetic human ANP and synthetic human BNP, as well as ANP combined with BNP, at concentrations corresponding to the concentration measured in each patient's plasma, were added to each well and incubated for 10 minutes. Afterwards, cells were washed once with PBS and lysed with 0.1M HCl. cGMP was then assayed using an ELISA (Enzo Life Sciences) as described elsewhere (Chen et al., Am. J. Physiol. Regul. Integr. Comp. Physiol. 314: R407-R414, 2018). Studies were repeated with human plasma with or without addition of the designer GC-A activator, MANP, at doses of 10⁻¹⁰ M and 10⁻⁸ M.

Statistical Analysis: Continuous variables are presented herein as median with interquartile range or mean with mean standard error, whereas categorical variables are presented herein as number and percentage. Linear regression was used for continuous characteristics, and logistic regression was used for binary characteristics. Continuous variables were compared using nonparametric Mann-Whitney tests. Categorical variables were statistically verified using chi-square tests. Correlations are presented herein as spearman ρ. All tests utilized two-sided p-values ≤0.05. Graphpad Prism 7.05 (San Diego, California) was the primary statistical software used for statistical analyses.

### Results

Study Populations: Baseline demographic data are presented in TABLE 2 to compare the healthy subjects (n=47) with the ADHF patients (n=107). Compared to the healthy subjects, ADHF patients were older, had higher BMI, and had lower eGFR and EF (all p<0.0001). Median NEP activity was lower in ADHF [12 (5.2, 21.2) nM/mL/min] compared to healthy subjects [32 (29.3, 35.7) nM/mL/min, p<0.0001]. ADHF patients presented with a history of hypertension, diabetes mellitus, atrial fibrillation, ischemic heart disease, myocardial infarction, hyperlipidemia, and stroke. Lastly, ADHF patients were taking, at admission, angiotensin-converting enzyme inhibitors, angiotensin II type 1 receptor blockers, beta blockers, loop diuretics, statins, and/or sacubitril/valsartan.

Natriuretic Peptides in Healthy Subjects and ADHF Patients: As shown in TABLE 2, median levels of ANP, BNP, NT-proANP, and NT-proBNP were all significantly elevated (p<0.001) in the ADHF patients compared to healthy subjects. BMI did not correlate with ANP (ρ=−0.21, p=0.16) or NT-proANP (ρ=−0.26, p=0.08) within the healthy subjects, but BMI did correlate with BNP (ρ=−0.37, p=0.01) and NT-proBNP (ρ=−0.32, p=0.03). In ADHF, however, ANP was inversely correlated with BMI (ρ=−0.49, p<0.0001) and EF (ρ=−0.30, p=0.002), while NT-proANP also was inversely correlated with BMI (ρ=−0.37, p<0.0001) and EF (ρ=−0.21, p=0.03). BNP similarly was inversely correlated with BMI (ρ=−0.53, p<0.0001) and EF (ρ=−0.40, p<0.0001) while NT-proBNP also inversely correlated with both BMI (ρ=−0.40, p<0.0001) and EF (ρ=−0.26, p=0.01) in ADHF.

TABLE 2
Baseline Characteristics and in Healthy Subjects and ADHF Patients

| | Healthy (n = 47) | | ADHF (n = 107) | | P |
|---|---|---|---|---|---|
| Male, n (%) | 25 | (53) | 74 | (69) | 0.06 |
| Age, (yr) | 52.0 | (44, 61) | 72 | (62, 81) | <0.0001 |
| ANP, pg/mL | 15.7 | (12, 20.9) | 118.1 | (37.8, 272.7) | <0.0001 |
| BNP, pg/mL | 23.0 | (16.0, 40) | 515.0 | (267, 1011) | <0.0001 |
| NTproANP, pg/mL | 60.1 | (38.2, 85.8) | 651.6 | (197.4, 846.2) | <0.0001 |
| NTproBNP, pg/mL | 46.0 | (27, 91) | 3,330 | (1538, 6764) | <0.0001 |
| cGMP, pmol/mL | 7.9 | (6.8, 10.6) | 13.3 | (9.4, 18.6) | <0.0001 |
| NEP Activity, nM/mL/min | 32 | (29.3, 35.7) | 12 | (5.2, 21.2) | <0.0001 |
| BMI, kg/m² | 27.6 | (25.1, 30.5) | 33.5 | (28, 38.4) | <0.0001 |
| eGFR, mL/min/1.73 m² | 82.3 | (73.1, 94.8) | 47.1 | (31, 68.6) | <0.0001 |

| | | | |
|---|---|---|---|
| EF, (%) | — | 38 | (25, 55) |
| Hypertension, n (%) | — | 87 | (81%) |
| Diabetes Mellitus, n (%) | — | 49 | (46%) |
| Atrial Fibrillation, n (%) | — | 80 | (75%) |
| IHD, n (%) | — | 74 | (69%) |
| MI, n (%) | — | 42 | (39%) |
| Hyperlipidemia, n (%) | — | 76 | (71%) |
| Stroke, n (%) | — | 26 | (24%) |
| ACE Inhibitor, n (%) | — | 37 | (35%) |
| ARB, n (%) | — | 14 | (13%) |
| Beta-blocker, n (%) | — | 68 | (64%) |
| Loop Diuretic, n (%) | — | 101 | (94%) |
| Statin, n (%) | — | 62 | (58%) |
| Sacubitril/Valsartan, n (%) | — | 6 | (6%) |

Values are presented as median (interquartile range) or n %.

IHD, ischemic heart disease; MI, myocardial infarction; ACE inhibitor, angiotensin-converting enzyme inhibitor, ARB, angiotensin II receptor blocker.

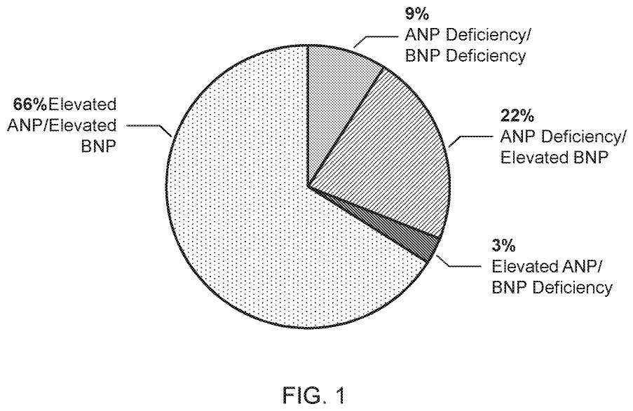

Analysis of Normal and Elevated NP Levels in ADHF Patients: Based on the ANP and BNP cutoffs described above, ANP was deficient in 31% (n=33) of ADHF patients and elevated in 69% (n=74) of ADHF patients, while BNP was in the normal (deficient) range in 12% (n=13) of ADHF patients and elevated in 88% (n=94) of ADHF patients (FIG. 1). 9% (n=10) of ADHF patients were deficient in both ANP and BNP, while 66% (n=70) of patients had elevated levels of both ANP and BNP. Three patients presented with elevated ANP but normal BNP. cGMP levels were significantly higher (p=0.001) in the ADHF High ANP and BNP cohort when compared with the ADHF deficient ANP and BNP cohort. BMI was lower (p<0.001) in the ADHF Elevated ANP and BNP cohort compared with the ADHF low ANP and BNP cohort.

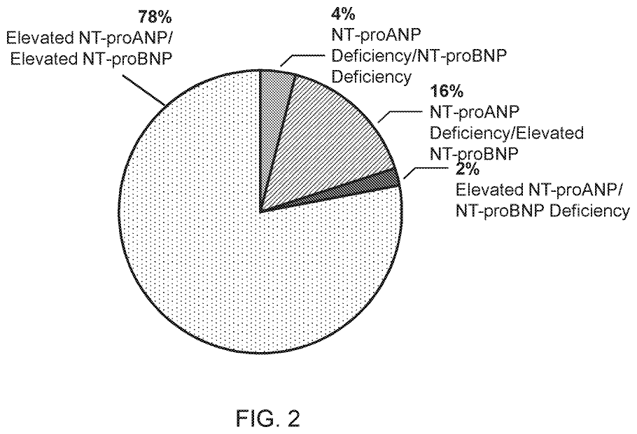

Based on the NT-proANP and NT-proBNP cutoffs described above, NT-proANP was deficient in 20% (n=21) of ADHF patients and elevated in 80% (N=86) (FIG. 2). 4% (N=4) of ADHF patients were deficient in both NT-proANP and NT-proBNP, while 16% were deficient in NT-proANP and had elevated NT-proBNP. Further, 78% (N=84) of ADHF patients had elevated levels of both NT-proANP and NT-proBNP, while 2% had elevated NT-proANP but were deficient in NT-proBNP.

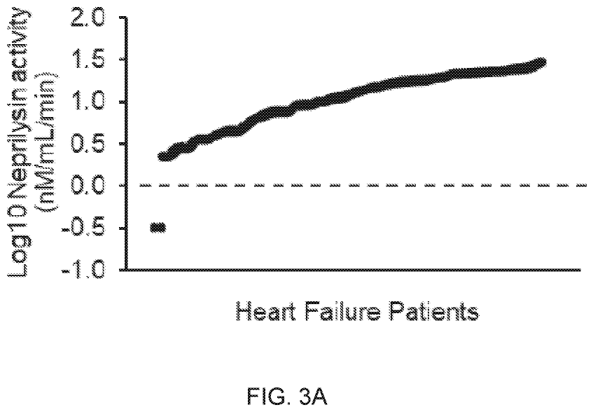

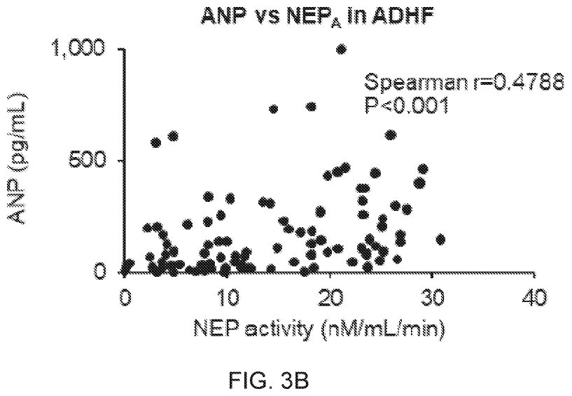

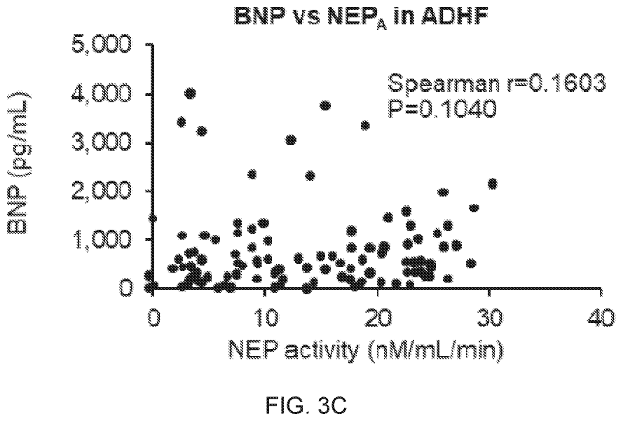

Neprilysin Activity in ADHF Patients: As illustrated in FIG. 3A, ANP levels were positively associated with NEP activity (ρ=0.48, P<0.001) (demonstrated through heat map visualization and plotted). As shown in FIGS. 3B and 3C, higher ANP, but not BNP, was associated with higher NEP activity, as BNP did not significantly correlate with NEP activity (ρ=0.16, p=0.104) (noting that FIG. 3B excludes four points that were outside the axis.)

NP/GC-A/cGMP Potency Assay with Human Plasma: As shown in FIG. 4, the ANP (25.5±4.8 pg/mL) and BNP (27±5.7 pg/mL) in healthy subject plasma generated a mean cGMP level of 10.3±0.7 pmol/mL. Synthetic ANP and BNP concentrations mimicking the endogenous ANP and BNP concentrations (25.5 and 27 pg/mL, respectively) found in healthy subject plasma produced mean cGMP levels of 7.8±0.5 pmol/mL and 4.7±0.4 pmol/mL cGMP respectively, while combined synthetic ANP+BNP produced 10.5±1.2 pmol/ml of cGMP. The synthetic ANP tended to be more potent than the synthetic BNP, while the synthetic BNP demonstrated significantly lower cGMP activation than normal human plasma.

Plasma from ADHF deficient ANP and BNP patients had an ANP level of 16.8±4.4 pg/mL and a BNP level of 47.4±7.5 pg/mL. Plasma from this cohort generated an average cGMP concentration of 9.5±0.7 pmol/mL (FIG. 4), which was comparable to healthy plasma, while synthetic ANP and BNP individually produced mean cGMP levels of 14.3±3.7 pmol/mL and 6.9±0.9 pmol/mL, respectively. Combined synthetic ANP+BNP produced 15.7±3.5 pmol/mL of mean cGMP, which was significantly greater than the cGMP produced from the plasma of the deficient ANP and BNP ADHF patients.

Plasma from ADHF patients with markedly elevated ANP and BNP had an ANP level of 350±57.2 pg/mL and a BNP level of 1225.7±290.1 pg/mL, which produced a mean cGMP concentration of 23.5±3.1 pmol/mL-a level that was significantly greater than that produced in response to healthy subject plasma and ANP deficient plasma. Synthetic ANP and BNP individually produced 59.7±13.8 pmol/mL and 80.3±16.9 pmol/mL of cGMP, respectively. Combined synthetic ANP+BNP together produced a mean cGMP concentration of 117.5±20.3 pmol/mL, which was the highest level of cGMP observed in the potency assay and was significantly greater than the cGMP produced with plasma from the elevated ANP/BNP ADHF cohort.

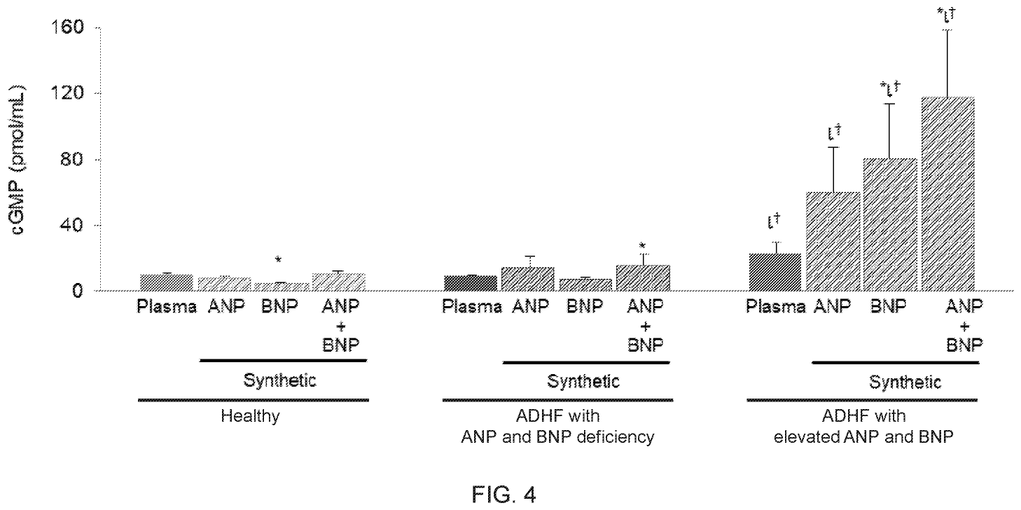

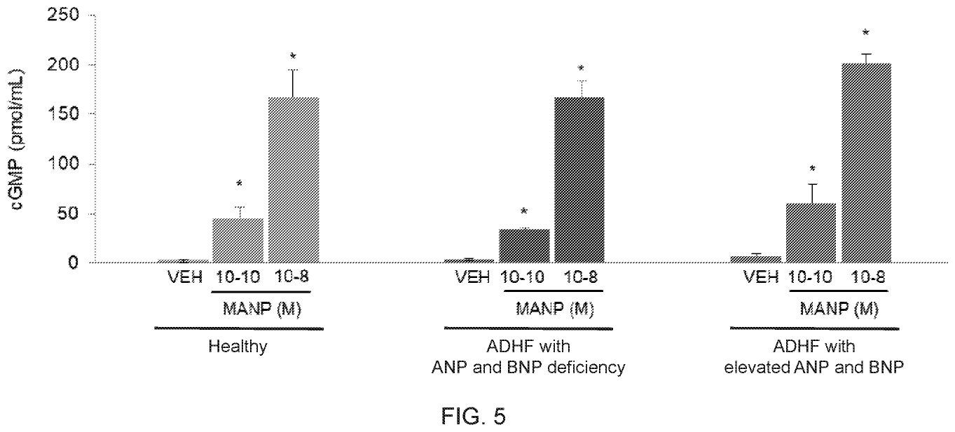

NP/GC-A/cGMP Potency of the MANP GC-A Agonist: To rescue the impaired NP response in ADHF, the efficacy of a GC-A agonist (MANP) was assessed. Specifically, the ability of MANP to elevate cGMP was assessed and compared to that for the plasma from healthy subjects, plasma from ADHF patients deficient in both ANP and BNP, and plasma from ADHF patients with elevated ANP and BNP (FIG. 5). Addition of MANP significantly elevated the healthy plasma mean cGMP levels from baseline at 3.1±0.2 pmol/mL to 45±5.5 pmol/mL (when treated with 10⁻¹⁰ M MANP) and 167±14.1 pmol/mL (when treated with 10⁻⁸ M MANP) (p<0.03 for both). Similarly, baseline cGMP levels obtained with the plasma of deficient ANP and BNP ADHF patients (3.6±0.8 pmol/mL) and elevated ANP and BNP ADHF patients (7.20.9±pmol/mL) were significantly increased (p<0.03) when the cells were treated with 10⁻¹⁰ M MANP (33.8±1.4 pmol/mL and 60.4±9.7 pmol/mL, respectively) and 10⁻⁸ M MANP (166.9±9.1 pmol/mL and 200.8±5.4 pmol/mL, respectively).

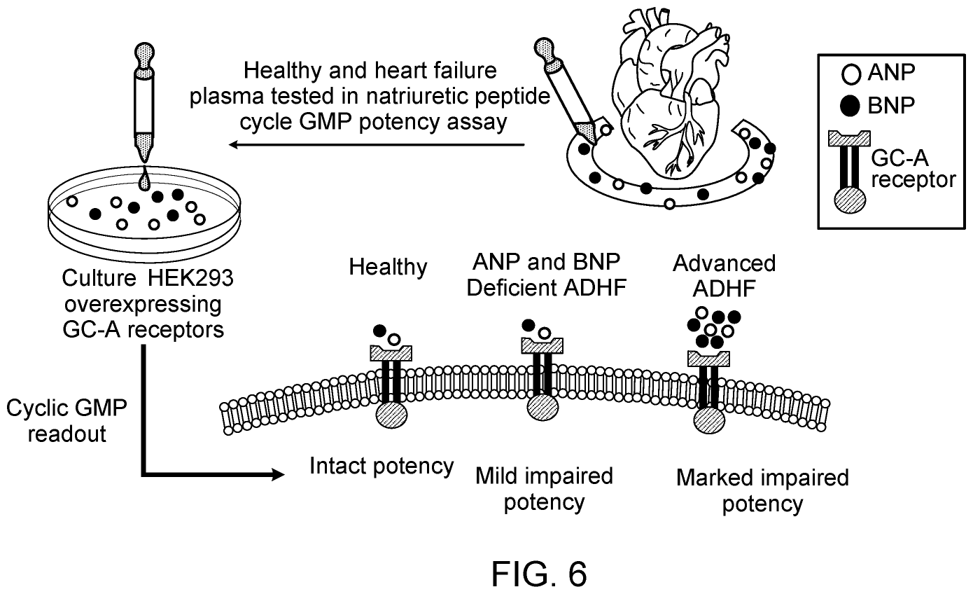

In summary, the studies described above assessed the biological activity of ANP and BNP from the plasma of healthy subjects and ADHF patients by measuring the production of the second messenger, cGMP, as a readout to the activation of GC-A, in comparison to cGMP produced from the synthetic ANP and BNP, and ANP+BNP. Healthy plasma demonstrated similar production of cGMP in comparison with synthetic ANP with BNP cGMP production, indicating that the biological production of cGMP is intact in healthy subjects, and suggesting that the NPs are preserved in their active form within the plasma and are reflective of a functional physiological state. These studies also demonstrated that the cardiac NP/GC-A/cGMP system is impaired in heart failure through a reduction in peptide potency and subsequent impaired GC-A/cGMP activation (FIG. 6), despite marked ANP and BNP peptide elevation. This cardiac endocrinopathy in ADHF indicates that NP replacement therapy with a GC-A activator such as MANP is useful to rescue the impaired NP state in HF in the presence of both cardiac NP deficiencies and impaired endogenous peptide potency.

### Example 2—A Small Molecule GC-A Receptor Positive Allosteric Modulator

A cell based high throughput screening (HTS) campaign of the NIH Molecular Libraries Small Molecule Repository was used to identify a small molecule GC-A positive allosteric modulator (PAM) scaffold. Further medicinal chemistry SAR efforts of the lead scaffold resulted in the development of a potent GC-A PAM, referred to herein as MCUF-651 (FIG. 7A).

### Method

Ex Vivo MCUF-651 Therapeutic Potency Assay with Human Plasma from Normal Subjects and Patients with Hypertension and Heart Failure: Stored human plasma samples from normal subjects and patients with HT and HF were utilized. The details of the recruitment of these participants were as described elsewhere (Reginauld et al., supra; and Ferrario et al., Hypertension 77:882-890, 2021). From all cohorts, plasma ANP was determined by an ANP radioimmunoassay, while plasma BNP was measured using a 2-site immunoenzymatic sandwich assay (Biosite Inc, Alere, France) (Murphy et al., JACC Heart Fail 9:127-136, 2021; Reginauld et al., supra; and Burnett et al., supra). HEK293 overexpressing human GC-A were cultured and grown as described above. Cells were grown in 48-well plates to 80-90% confluence. On the day of the experiment, cells were pre-incubated with MCUF-651 at doses of 1, 5, or 10 μM or with vehicle (Veh) in 250 μL treatment buffer (as described above) for 5 minutes at 37° C. 25 μL of human plasma was then added and incubated for additional 10 minutes. Cells were then washed with PBS once and lysed with 0.1 M HCl, and intracellular cGMP levels were measured in the lysate using a commercial cGMP ELISA kit (Enzo Life Sciences, Farmingdale, NY) as instructed by the manufacturer.

Statistical Analysis: Data are expressed as mean±SEM, unless stated otherwise. Unpaired t-test was performed for comparison between groups in therapeutic efficacy assay studies.

### Results

Ex Vivo MCUF-651 Potency in Human Plasma: To further define the therapeutic potential of MCUF-651, an ex vivo potency assay that utilized human plasma from normal subjects and from patients with hypertension and HF (N=6 per group), which had various levels of circulating ANP and BNP. TABLE 3 reports subject characteristics and their respective plasma ANP and BNP levels. As illustrated in FIG. 7B, MCUF-651 potentiated the generation of cGMP levels in HEK293 GC-A cells in all three cohorts. MCUF-651 demonstrated the greatest cGMP potency in HF plasma in which circulating levels of ANP and BNP were the highest. These ex vivo findings provided validation that MCUF-651 possesses GC-A enhancing action in human plasma and operates in a PAM mode, with increasing cGMP generation in association with increasing concentrations of endogenous ANP and BNP.

TABLE 3
Human Subject and Patient Characteristics

| | | Hypertensive | |
|---|---|---|---|
| | Normal Subjects | Patients | HF Patients |
| | (n = 6) | (n = 6) | (n = 6) |
| Age, years | 59 ± 8 | 69 ± 9 | 65 ± 10 |
| Sex, female (%) | 75% | 50% | 75% |
| BMI, kg/m² | 28 ± 3 | 25 ± 1 | 29 ± 5 |
| eGFR, mL/min/1.73 m² | 75 ± 15 | 70 ± 8 | 50 ± 25 |
| ANP, pg/mL | 26 ± 12 | 13 ± 8 | 350 ± 140 |
| BNP, pg/mL | 27 ± 14 | 59 ± 37 | 1226 ± 711 |

Values are presented as mean ± SD, n (%).

These studies demonstrated that MCUF-651 enhances the ability of endogenous ANP and BNP in the plasma of normal subjects and patients with HT or HF to generate GC-A mediated cGMP ex vivo. Thus, this work demonstrates the discovery and development of a first-in-class, oral, small molecule GC-A PAM that holds great potential as a novel therapeutic for cardiovascular, renal, and metabolic diseases.

### Example 3—Assessing and Treating a Human Likely to Respond to a pGC Stimulator

A plasma sample is obtained from a human having a cardiovascular disease such as ADHF. A portion of the plasma sample is added to a first population of cells (e.g., HEK293 cells) that overexpress pGC-A, pGC-B, or both pGC-A and pGC-B, and the cells are incubated in the presence of the plasma sample for about 10 minutes. A second population of the cells overexpressing pGC-A and/or pGC-B is contacted with a second portion of the plasma sample in combination with a pGC receptor stimulator (e.g., MANP). The second population of cells is incubated with the plasma sample and the pGC stimulator for about 10 minutes. The first and second populations of cells are lysed and the cGMP concentration in each is determined (e.g., using an ELISA), to give a first cGMP concentration for the plasma-only incubated cells and a second cGMP concentration for the cells incubated with plasma and pGC stimulator. The second cGMP concentration is compared to the first cGMP concentration, and the second cGMP concentration is determined to be at least 5% greater than the first cGMP concentration. The human is classified as being likely to respond to treatment with the pGC stimulator and/or is administered a dose of the pGC stimulator.

### Example 4—Assessing and Treating a Human not Likely to Respond to a pGC Stimulator

A plasma sample is obtained from a human having a cardiovascular disease such as ADHF. A portion of the plasma sample is added to a first population of cells (e.g., HEK293 cells) that overexpress pGC-A, pGC-B, or both pGC-A and pGC-B, and the cells are incubated in the presence of the plasma sample for about 10 minutes. A second population of the cells overexpressing pGC-A and/or pGC-B is contacted with a second portion of the plasma sample in combination with a pGC receptor stimulator (e.g., MANP). The second population of cells is incubated with the plasma sample and the pGC stimulator for about 10 minutes. The first and second populations of cells are lysed and the cGMP concentration in each is determined (e.g., using an ELISA), to give a first cGMP concentration for the plasma-only incubated cells and a second cGMP concentration for the cells incubated with plasma and pGC stimulator. The second cGMP concentration is compared to the first cGMP concentration, and the second cGMP concentration is determined not to be at least 5% greater than the first cGMP concentration. The human is classified as not being likely to respond to treatment with the pGC stimulator and is not administered a dose of the pGC stimulator.

### Example 5—Ex Vivo Potency Assay to Define Therapeutic Potential via Production of CGMP

An ex vivo assay was developed to define therapeutic potential via the production of cGMP. Briefly, the assay tested the cGMP response of human plasma from patients with CKD, HT and ADHF, either (1) alone or (2) in combination with a native natriuretic peptide (ANP or CNP), a designer natriuretic peptide (MANP, C53 or CRRL-408), or a small molecule pGC positive allosteric modulator (MCUF-651), in HEK293 cells that overexpress the human pGC-A or pGC-B receptor.

### Methods

Human Participants: Stored human plasma samples from normal subjects and patients with HT and HF were utilized. Stored human plasma samples from patients with HT (n=6), ADHF (n-6), or CKD (n=5) were utilized. The details of the recruitment of HT and ADHF participants were as described elsewhere (Reginauld et al., supra; Ferrario et al., supra; and Ma et al., JACC Heart Fail. 9(9):613-623, 2021). CKD patients were to have a stable estimated glomerular filtration rate (eGFR) between 15 and 59 ml/min/1.73 m² in the year prior to recruitment, no history of heart disease, and no active cancer or history of amyloid disease.

### Native and Designer Natriuretic Peptide Amino Acid Sequences

MANP (designer pGC-A activator):
(SEQ ID NO: 1)
SLRRSSCFGGRMDRIGAQSGLGCNSFRYRITAREDKQGWA
ANP (native/endogenous pGC-A activator):
(SEQ ID NO: 2)
SLRRSSCFGGRMDRIGAQSGLGCNSFRY

CRRL-408 (designer dual pGC-A and pGC-B activator containing, in order from N-terminus to C-terminus, the C-terminal 8 amino acids of the NT-CNP53 polypeptide, the mature CNP sequence (italicized), and the C-terminal tail of Dendroaspis natriuretic peptide (DNP)): KYKGANKKGLSKGCFGLKLDRIGSMSGLGCPSLRDPRPNAPSTSA (SEQ ID NO:3)

CNP (native/endogenous pGC-B) activator:
(SEQ ID NO: 4)
GLSKGCFGLKLDRIGSMSGLGC

C53 (designer pGC-B activator that is the 53 amino acid intermediate form of CNP and results from intracellular cleavage of the CNP prohormone by the enzyme, furin): DLRVDTKSRAAWARLLQEHPNARKYKGANKKGLSKGCFGLKLDRIG SMSGLGC (SEQ ID NO:5)

Plasma ANP, BNP and CNP Levels: From all cohorts, plasma ANP was determined by an ANP radioimmunoassay, plasma BNP was measured using a 2-site immunoenzymatic sandwich assay (Biosite Inc., Alere, France), and plasma CNP was determined by a nonequilibrium radioimmunoassay (Murphy et al., supra; Reginauld et al., supra; Burnett et al., supra; and Ma et al., supra).

In vitro Cell Culture: HEK 293 cells were stably transfected with human pGC-A or pGC-B using LIPOFECTAMINE™ (Invitrogen, Grand Island, NY) (Chen et al. 2020, supra). Transfected cells (HEK/GC-A or (HEK/GC-B) were maintained in DMEM supplemented with 10% FBS and 250 μg/ml G418. Cells in 48-well plates were grown to 80-90% confluency and then incubated in Hanks Balanced Salt Solution (HBSS, Invitrogen) containing 20 mM HEPES, 0.1% BSA, and 0.5 mM 3-isobutyl-1-methylxanthine (IBMX, Sigma).

NP/pGC-A/cGMP Potency Assay: Human plasma samples from patients with HT (N=6), ADHF (N=6), and CKD (N=5) were utilized. Stably transfected HEK293 cells engineered to overexpress human pGC-A were cultured (as described above) and were grown in 48-well plates to 80-90% confluence. On the day of the experiment, human plasma was diluted 10× with HBSS. Diluted plasma (250 μl) was added together with vehicle (PBS), native ANP, MANP, or CRRL-408 at doses of 10⁻¹⁰, 10⁻⁸, or 10⁻⁷ M to the HEK/pGC-A cells and incubated for 10 minutes at 37° C. Afterwards, the cells were washed once with PBS and lysed with 0.1M HCl. cGMP was then assayed using an ELISA (Enzo Life Sciences) as described elsewhere (Chen et al. 2018, supra).

MCUF-651/NP/pGC-A/cGMP Potency Assay: Human plasma samples from patients with HT (N=6), ADHF (N=6), and CKD (N=5) were utilized. The details of the recruitment of participants are described above. Stably transfected HEK293 cells engineered to overexpress human pGC-A were cultured (as described above) and grown in 48-well plates to 80-90% confluence. On the day of the experiment, human plasma was diluted 10× with HBSS. 250 μl of diluted plasma was added together with vehicle (DSMO) or the MCUF-651 small molecule pGC-A positive allosteric modulator at doses of 1, 5, or 10 μM to the HEK/pGC-A cells and incubated for 10 minutes at 37° C. Cells were then washed with PBS once and lysed with 0.1 M HCl, and intracellular cGMP levels were measured in the lysate using a commercial cGMP ELISA kit (Enzo Life Sciences, Farmingdale, NY) as instructed by the manufacturer.

NP/pGC-B/cGMP Potency Assay: Human plasma samples from patients with ADHF (N=6) were utilized. Stably transfected HEK293 cells engineered to overexpress human pGC-B were cultured (as described above) and grown in 48-well plates to 80-90% confluence. On the day of the experiment, human plasma was diluted 10× with HBSS. Diluted plasma (250 μl) was added together with vehicle (PBS), native CNP (an endogenous pGC-B activator), C53 (a designer CNP analog/pGC-B activator) or CRRL-408 (a designer dual pGC-A and pGC-B activator) at doses of 10⁻¹⁰, 10⁻⁸, or 10⁻⁷ M to the HEK/GC-B cells and incubated for 10 minutes at 37° C. Afterwards, cells were washed once with PBS and lysed with 0.1M HCl. cGMP was then assayed using an ELISA (Enzo Life Sciences) as described elsewhere (Chen et al. 2018, supra).

### Results

An ex vivo assay was developed to evaluate the therapeutic potential of various pGC targets, via the production of cGMP. The assay tested the cGMP response of human plasma from patients with CKD (n=5), HT (n=6), and ADHF (n=6), either (1) alone or (2) in combination with native natriuretic peptide (ANP or CNP), designer natriuretic peptide (MANP, C53 or CRRL-408), or small molecule pGC positive allosteric modulator (MCUF-651) in HEK293 cells that overexpressed the human pGC-A or pGC-B receptor. TABLE 4 reports the age, sex, and circulating ANP, BNP, and CNP levels for the study subjects.

TABLE 4
Human Patient Characteristics*

| | CKD Patients | HT Patients | ADHF Patients |
|---|---|---|---|
| | (n = 5) | (n = 6) | (n = 6) |
| Age, years | 64 ± 8 | 68 ± 10 | 78 ± 7 |
| Sex, female (%) | 40% | 67% | 33% |
| ANP, pg/mL | 40 ± 32 | 25 ± 13 | 352 ± 368 |
| BNP, pg/mL | 138 ± 148 | 46 ± 35 | 538 ± 570 |
| CNP, pg/mL | 16 ± 11 | 23 ± 12 | 27 ± 12 |

Values are presented as mean ± SD, n (%).

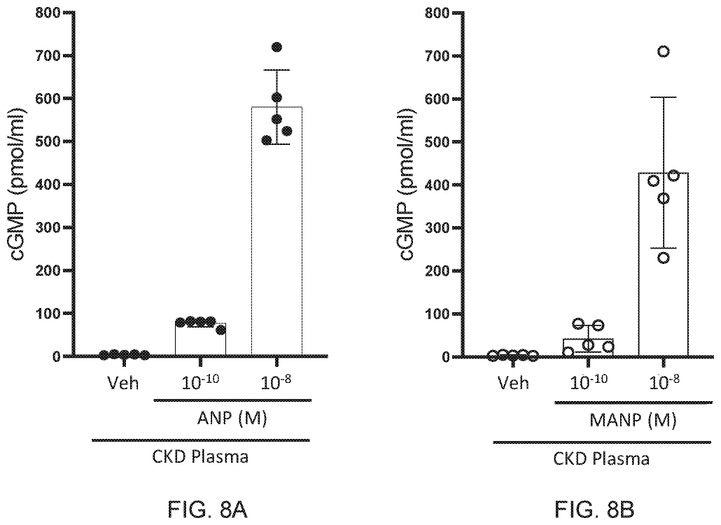

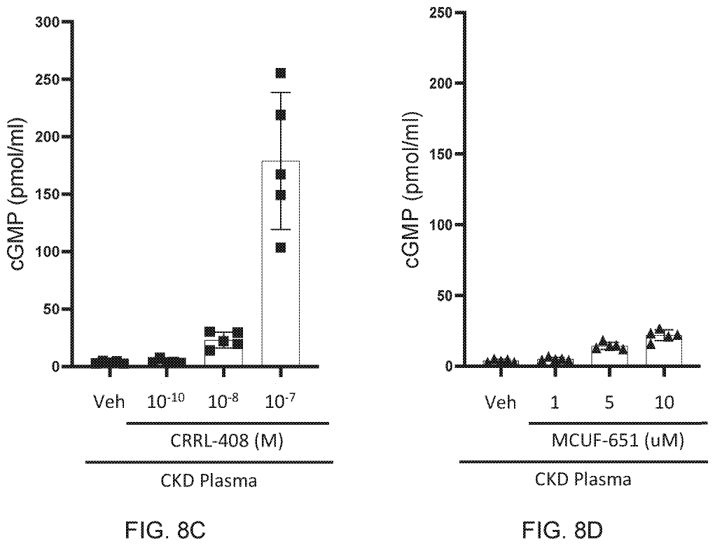

As illustrated in FIGS. 8A-8C, cGMP levels were modestly potentiated when CKD plasma was treated with ANP or MANP at a dose of 10⁻¹⁰ M (FIGS. 8A and 8B), and with CRRL-408 at a dose of 10⁻⁸ M (FIG. 8C) in HEK293 pGC-A cells. cGMP levels were markedly potentiated when CKD plasma was treated with ANP or MANP at a dose of 10⁻⁸ M (FIGS. 8A and 8B), and with CRRL-408 at a dose of 10⁻⁷ M (FIG. 8C) in HEK293 pGC-A cells. Interestingly, there was a variable cGMP response with MANP treatment at 10⁻⁸ M and CRRL-408 treatment at 10⁻⁷ M, thus demonstrating the differential responsiveness to these designer natriuretic peptide treatments in CKD patients. Furthermore and unexpectedly, cGMP levels were only minimally potentiated when CKD plasma was treated with MCUF-651 alone at doses of 5 and 10 μM (FIG. 8D), with no response at a dose of 1 μM (FIG. 8D) in HEK293 pGC-A cells.

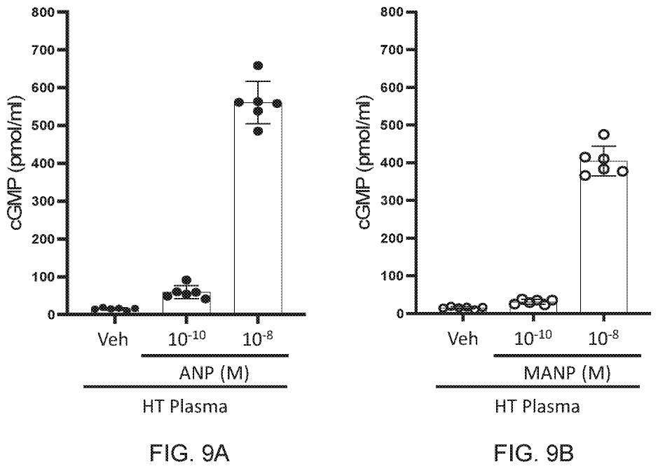

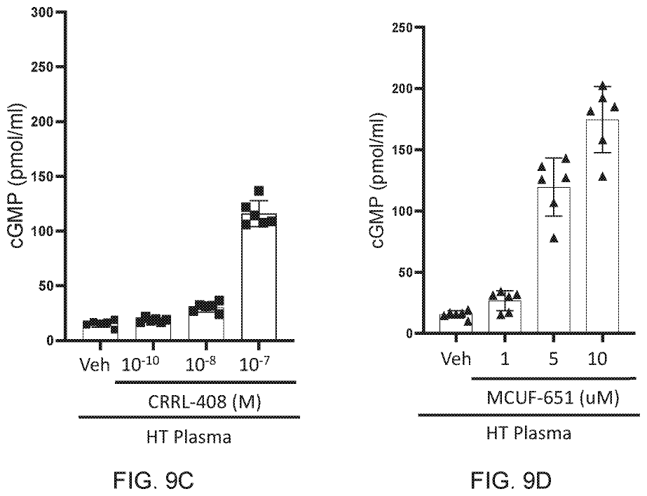

As illustrated in FIGS. 9A-9C, cGMP levels were modestly potentiated when HT plasma was treated with ANP and MANP at a dose 10⁻¹⁰ M (FIGS. 9A and 9B), and with CRRL-408 at a dose of 10⁻⁸ M (FIG. 9C), in HEK293 pGC-A cells. cGMP levels were markedly potentiated when HT plasma was treated with ANP and MANP at a dose 10⁻⁸ M (FIGS. 9A and 9B), and with CRRL-408 at a dose of 10⁻⁷ M (FIG. 9C), in HEK293 pGC-A cells. Notably, cGMP levels were modestly potentiated when HT plasma was treated with MCUF-651 at a dose of 1 μM (FIG. 9D), and were markedly potentiated when treated with MCUF-651 at doses of 5 and 10 μM (FIG. 9D) in HEK293 pGC-A cells.

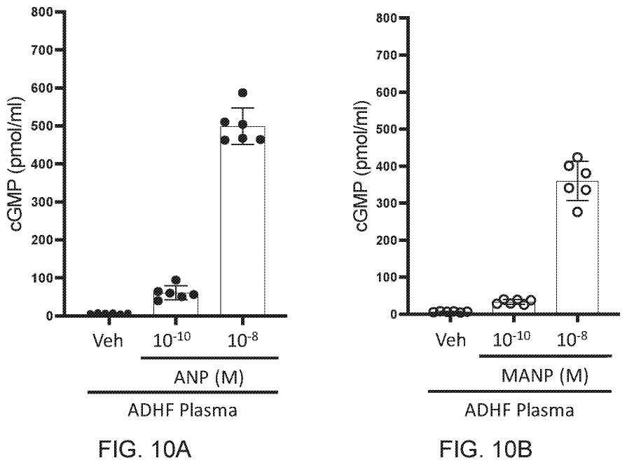

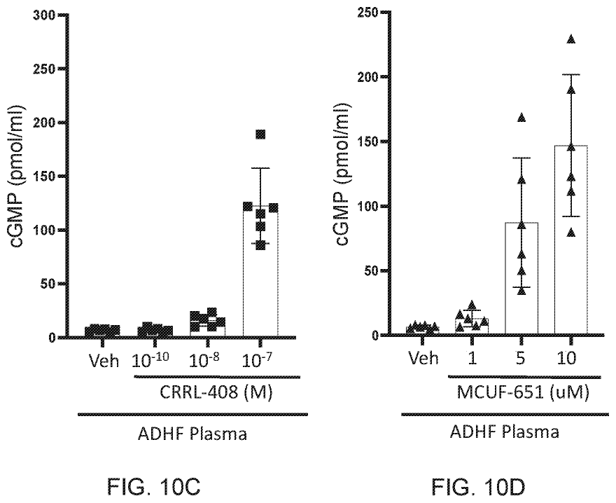

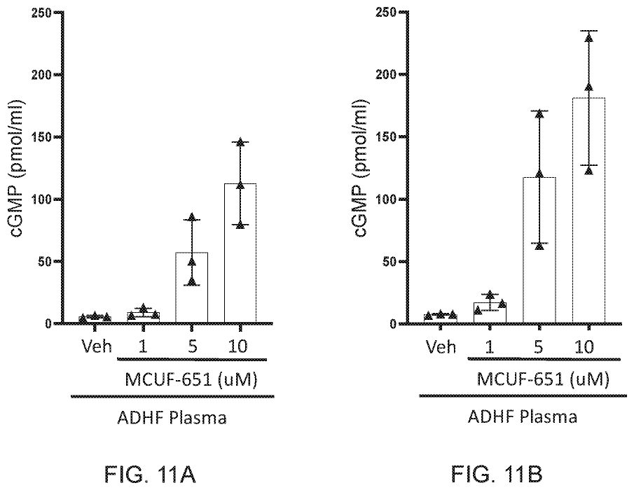

As illustrated in FIGS. 10A-10C, cGMP levels were modestly potentiated when ADHF plasma was treated with ANP and MANP at a dose 10⁻¹⁰ M (FIGS. 10A and 10B), and with CRRL-408 at a dose of 10⁻⁸ M (FIG. 10C), in HEK293 GC-A cells. cGMP levels were markedly potentiated when ADHF plasma was treated with ANP and MANP at a dose 10⁻⁸ M (FIGS. 10A and 10B), and with CRRL-408 at a dose of 10⁻⁷ M (FIG. 10C), in HEK293 pGC-A cells. Interestingly, there was a single ADHF patient that had a greater cGMP response with CRRL-408 treatment at 10⁻⁷ M dose, thus demonstrating the potential of a differential response to CRRL-408 in ADHF patients. Notably, cGMP levels were modestly potentiated when ADHF plasma was treated with MCUF-651 at a dose of 1 μM (FIG. 10D), and were markedly potentiated when treated with MCUF-651 at doses of 5 and 10 μM (FIG. 10D) in HEK293 pGC-A cells. Importantly, there was a variable cGMP response with MCUF-651 at doses of 5 and 10 μM (FIG. 10D), which in part may be attributable to levels of circulating ANP and BNP. Thus, the cGMP response to MCUF-651 in ADHF plasma was evaluated based on low or high circulating ANP and BNP (FIGS. 11A-11B). As illustrated in FIG. 11A, MCUF-651 demonstrated lower cGMP potency, particularly at the 5 and 10 μM doses, in ADHF plasma in which circulating ANP and BNP levels were lower. In contrast, FIG. 11B illustrates the higher cGMP potency with MCUF-651, particularly at the 5 and 10 μM doses, in ADHF plasma in which circulating ANP and BNP levels were higher.

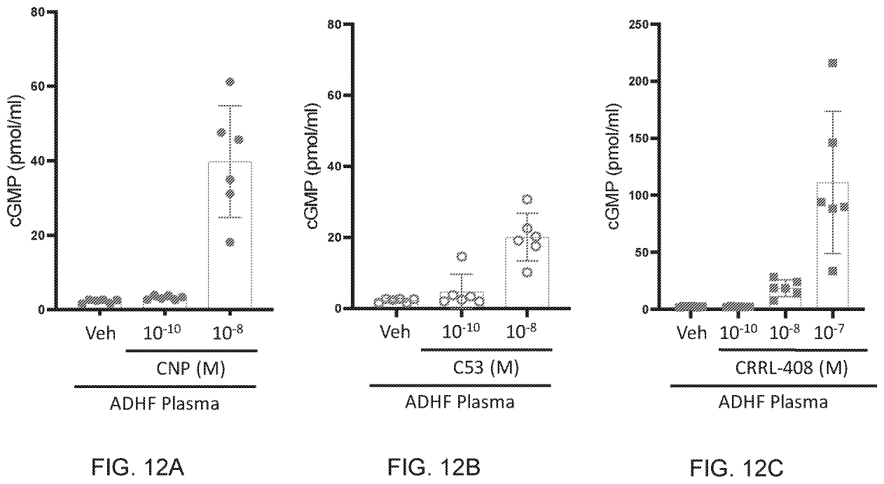

As shown in FIGS. 12A-12C, cGMP levels were modestly potentiated in ADHF plasma in a single patient when treated with C53 at a dose of 10⁻¹⁰ M (FIG. 12B), and in ADHF plasma of all patients when treated with CRRL-408 at a dose of 10⁻⁸ M (FIG. 12C), in HEK293 pGC-B cells. cGMP levels were markedly potentiated, however, with a variable responsiveness, when ADHF plasma was treated with CNP or C53 at a dose of 10⁻⁸ M (FIGS. 12A and 12B), and with CRRL-408 at a dose of 10⁻⁷ M (FIG. 12C), in HEK293 pGC-B cells.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for determining whether a mammal having a cardiovascular, cardiorenal, or cardiometabolic disorder is likely to be responsive to treatment with a particulate guanylate cyclase (pGC) receptor stimulator, said method comprising:
(a) contacting, with a first biological fluid sample from said mammal, a first population of cells that express a pGC-A receptor, a pGC-B receptor, or both a pGC-A receptor and a pGC-B receptor, and measuring a first amount of cGMP produced by said first population of cells after said contacting;
(b) contacting a second population of said cells that express a pGC-A receptor, a pGC-B receptor, or both a pGC-A receptor and a pGC-B receptor, with a second biological fluid sample from said mammal in combination with said pGC receptor stimulator, and measuring a second amount of cGMP produced by said second population of cells after said contacting;
(c) comparing said first amount of cGMP to said second amount of cGMP; and
(d) when said second amount of cGMP is at least 5% greater than said first amount of cGMP, classifying said mammal as likely to respond to said pGC receptor stimulator, or when said second amount of cGMP is not at least 5% greater than said first amount of cGMP, classifying said mammal as not likely to respond to treatment with said pGC receptor stimulator.

2. The method of claim 1, wherein said mammal is a human.

3. The method of claim 1, wherein said cardiovascular, cardiorenal, or cardiometabolic disorder comprises heart failure (HF), hypertension (HT), cardiorenal syndrome, chronic kidney disease, metabolic syndrome, acute kidney injury, acute kidney disease, or cardiomyopathy.

4. The method of claim 3, wherein said HF is acute decompensated heart failure (ADHF), HF with reduced ejection fraction, or HF with preserved ejection fraction, or wherein said HT is primary HT, uncontrolled HT, resistant HT, or pulmonary HT.

5. (canceled)

6. The method of claim 1, wherein said biological fluid sample is a blood sample or a plasma sample.

7. The method of claim 1, comprising incubating said first and second populations of cells with said first and second biological fluid samples for at least 10 minutes before measuring said first and second amounts of cGMP.

8. The method of claim 1, wherein said pGC receptor stimulator is selected from the group consisting of MANP, C53, CRRL-408, MCUF-651, vosoritide, nesiritide, carperitide, cenderitide, and neprilysin inhibitors.

9. A method for treating a mammal having a cardiovascular, cardiorenal, or cardiometabolic disorder, said method comprising:
(a) contacting, with a first biological fluid sample from said mammal, a first population of cells that express a pGC-A receptor, a pGC-B receptor, or both a pGC-A receptor and a pGC-B receptor, and measuring a first amount of cGMP produced by said first population of cells after said contacting;
(b) contacting a second population of said cells that express a pGC-A receptor, a pGC-B receptor, or both a pGC-A receptor and a pGC-B receptor, with a second biological fluid sample from said mammal in combination with a pGC receptor stimulator, and measuring a second amount of cGMP produced by said second population of cells after said contacting;
(c) comparing said first amount of cGMP to said second amount of cGMP; and
(d) administering said pGC receptor stimulator to said mammal when said second amount of cGMP is at least 5% greater than said first amount of cGMP.

10. The method of claim 9, wherein said mammal is a human.

11. The method of claim 9, wherein said cardiovascular, cardiorenal, or cardiometabolic disorder comprises HF, HT, cardiorenal syndrome, chronic kidney disease, metabolic syndrome, acute kidney injury, acute kidney disease, or cardiomyopathy.

12. The method of claim 11, wherein said HF is ADHF, HF with reduced ejection fraction, or HF with preserved ejection fraction, or wherein said HT is primary HT, uncontrolled HT, resistant HT, or pulmonary HT.

13. (canceled)

14. The method of claim 9, wherein said biological fluid sample is a blood sample or a plasma sample.

15. The method of claim 9, comprising incubating said first and second populations of cells with said first and second biological fluid samples for at least 10 minutes before measuring said first and second amounts of cGMP.

16. The method of claim 9, wherein said pGC receptor stimulator is selected from the group consisting of MANP, C53, CRRL-408, MCUF-651, vosoritide, nesiritide, carperitide, cenderitide, and neprilysin inhibitors.

17. A method for treating a mammal having a cardiovascular, cardiorenal, or cardiometabolic disorder, said method comprising administering a pGC receptor stimulator to said mammal, wherein said mammal was identified being in need of said pGC receptor stimulator based on a method comprising:
(a) contacting, with a first biological fluid sample from said mammal, a first population of cells that express a pGC-A receptor, a pGC-B receptor, or both a pGC-A receptor and a pGC-B receptor, and measuring a first amount of cGMP produced by said first population of cells after said contacting;
(b) contacting a second population of said cells that express a pGC-A receptor, a pGC-B receptor, or both a pGC-A receptor and a pGC-B receptor, with a second biological fluid sample from said mammal in combination with said pGC receptor stimulator, and measuring a second amount of cGMP produced by said second population of cells after said contacting;
(c) comparing said first amount of cGMP to said second amount of cGMP; and
(d) determining that said second amount of cGMP is at least 5% greater than said first amount of cGMP.

18. The method of claim 17, wherein said mammal is a human.

19. The method of claim 17, wherein said cardiovascular, cardiorenal, or cardiometabolic disorder comprises HF, HT, cardiorenal syndrome, chronic kidney disease, metabolic syndrome, acute kidney injury, acute kidney disease, or cardiomyopathy.

20. The method of claim 19, wherein said HF is ADHF, HF with reduced ejection fraction, or HF with preserved ejection fraction, or wherein said HT is primary HT, uncontrolled HT, resistant HT, or pulmonary HT.

21. (canceled)

22. The method of claim 17, wherein said biological fluid sample is a blood sample or a plasma sample.

23. The method of claim 17, wherein said cells were incubated with said biological fluid sample for at least 10 minutes before measuring said amount of cGMP.

24. The method of claim 17, wherein said pGC receptor stimulator is selected from the group consisting of MANP, C53, CRRL-408, MCUF-651, vosoritide, nesiritide, carperitide, cenderitide, and neprilysin inhibitors.


SEQUENCE LISTING (5 sequences)

SEQ ID NO: 1 - protein, 40 aa, synthetic construct
SLRRSSCFGGRMDRIGAQSGLGCNSFRYRITAREDKQGWA

SEQ ID NO: 2 - protein, 28 aa, Homo sapiens
SLRRSSCFGGRMDRIGAQSGLGCNSFRY

SEQ ID NO: 3 - protein, 45 aa, synthetic construct
KYKGANKKGLSKGCFGLKLDRIGSMSGLGCPSLRDPRPNAPSTSA

SEQ ID NO: 4 - protein, 22 aa, Homo sapiens
GLSKGCFGLKLDRIGSMSGLGC

SEQ ID NO: 5 - protein, 53 aa, synthetic construct
DLRVDTKSRAAWARLLQEHPNARKYKGANKKGLSKGCFGLKLDRIGSMSGLGC